(19) United States
(12) Patent Application Publication
(10) Pub. No.: US 2010/0331747 A1
(43) Pub. Date: Dec. 30, 2010

(54) Protective cast cover for bathing

(75) Inventor: Cser Ferenc, Kaposvar (HU)

Correspondence Address: ROBERT M. DOWNEY, P.A., 6751 N. FEDERAL HWY., SUITE 300, BOCA RATON, FL, 33487, US

(73) Assignees: Laszlo Nagy; Imre Janotka

(21) Appl. No.: 12/459,325

(22) Filed: Jun. 29, 2009

(51) Int. Cl. A61F 5/00

(52) U.S. Cl. 602/3

(57) ABSTRACT

A cover is adapted to be worn over a user's limb to shield a bandage, dressing or cast from exposure to water. The cover includes a generally elongate body defined by a shell formed of a flexible, water resistant and air breathing material. The shell surrounds an interior of the cover, including a lower interior chamber and an upper interior chamber. An open top end communicates with the upper interior chamber and is sized and configured to permit passage of the user's limb and the cast, bandage or dressing therethrough and into the upper and lower interior chambers. A cinch strap with a releasable securing mechanism closes the open top end snug around the user's limb to provide a first barrier against water entry into the interior. An inner cuff separates the upper and lower interior chambers and includes an elastomeric opening that fits snug against the user's limb, above the cast, bandage or dressing, to provide a second barrier against water entry into the lower interior chamber.

### BACKGROUND OF THE INVENTION

1. Field of the Invention

The present invention relates to protective covers for bandages and casts and, more particularly, to a cover device that fits over a persons hand or foot and at least a portion of the length of the persons arm or leg to protectively cover the bandage or cast.

2. Discussion of the Related Art

When a person is fitted with a bandage or cast for healing a wound or fractured bone, it is important to keep the bandage or cast dry in order to prevent infection, as well as to avoid deterioration of the bandage or cast. However, this can be difficult, especially when attempting to bathe in a tub or shower. Exposure of a bandage or cast to moisture is also a threat when it rains or if the person is engaged in an activity, such as boating, which presents the possibility of getting wet.

In order to provide protection against water exposure, others have proposed various sleeves, bags and cover devices that fit over a portion of the body and a bandage or a cast worn on that body portion. These various protective devices in the related art are typically formed of a plastic material that provides a water resistant barrier.

Typically, the protective cast covers in the related art are secured to the user's body with the use of an elastic band or tape. This means of closing the protective cover over the bandage or cast has been found to be inadequate in preventing entry of water or moisture into the protective cover. Moreover, the water resistant material of these protective cover devices, while effective to prevent penetration of water therethrough, fail to allow for air flow or breathing between the interior and exterior of the cover. This traps body heat which causes a rapid temperature rise within the interior of the cover device, resulting in excess perspiration and high humidity that remains contained inside the cover device. Perspiration and moisture that enters the cover device, as a result of the inadequate closure means, combined with the trapped hot, moist air within the cover can lead to foul odors, rash, fungus, infection and deterioration of the bandage or cast.

Accordingly, there remains a need for a protective device that can be easily fitted over a portion of a persons body to cover a bandage or cast, and wherein the device is adapted to be effectively closed to prevent entry of water or moisture. There is a further need for a protective cover device that allows for breathing to prevent trapped heat and excessive perspiration.

### OBJECTS AND ADVANTAGES OF THE INVENTION

Considering the foregoing, it is a primary object of the present invention to provide a protective cover that fits over a portion of a person's body to cover a bandage or cast, and wherein the protective cover device is adapted to close against the user's body in a manner that prevents entry of water or moisture under the cover device.

It is a further object of the present invention to provide a protective cover device which is adapted to fit over a hand or foot and at least partially over a person's arm or leg, and wherein the protective cover device includes a first closure mechanism providing a first barrier against water entry and a second closure mechanism providing a second barrier of water entry, thereby substantially minimizing the possibility of exposing the bandage or cast to water or moisture.

It is still a further object of the present invention to provide a protective cover device for covering a bandage or cast on a person's body, and wherein the protective cover device is made at least partially of a breathable material that allows for passage of air between an interior of the device and an exterior of the device while preventing transfer of moisture therethrough.

It is still a further object of the present invention to provide a protective cover device for covering a portion of a person's body and a bandage or cast worn on the person's body, and wherein the protective cover device is adapted to be adjustably closed against the person's body to provide several barriers against entry of moisture or water.

It is still a further object of the present invention to provide a protective cover device that is adapted to be adjustably fitted over a portion of a person's body to cover a bandage or cast, and wherein the protective cover device is water resistant and breathable to protect against development of foul odors, rashes, fungus, infection and deterioration of the bandage or cast that can result from trapped heat and prolonged exposure to moisture or water.

These and other objects and advantages of the present invention are more readily apparent with reference to the following detailed description and accompanying drawings.

### SUMMARY OF THE INVENTION

The present invention is directed to a protective cover device that is adapted to be worn over a user's limb to shield a bandage, wound dressing or cast from exposure to water and moisture. The protective cover is generally in the form of an elongate bag that has a shell formed of a flexible, water resistant and air breathable material. The shell surrounds an interior that is sized and configured to comfortably receive a person's limb (i.e., hand and arm or foot and leg) as well as a bandage or cast that is worn on the person's limb. The interior of the cover device is divided between a lower interior chamber and an upper interior chamber. An open top end communicates with the upper interior chamber and is sized and configured to permit passage of the user's limb and the cast, bandage or dressing therethrough and into the upper and lower interior chambers. The open top end is closed snugly around the user's limb with the use of a cinch strap. The cinch strap is provided with releasable securing means for closing the open top end snug around the user's limb in order to provide a first barrier against water entry into the interior of the cover device. An inner cuff separates the upper and lower interior chambers. The inner cuff is secured to an inner surface of the shell of the cover device and surrounds a central opening that is fitted with an elastic band. The central opening of the cuff is adapted to permit passage of the user's hand or foot therethrough, as well as a portion of the limb and the bandage or cast, so that the bandage or cast is contained within the lower interior chamber. The elastic band sits snug against the user's limb, above the bandage or cast, to provide a second barrier against water entry into the lower interior chamber.

### BRIEF DESCRIPTION OF THE DRAWINGS

For a fuller understanding of the nature of the present invention, reference should be made to the following detailed description taken in conjunction with the accompanying drawings in which:

FIG. 1 is a front perspective view showing the protective cover device of the present invention fitted over the hand and arm of a person wearing a cast on their hand, wrist and forearm;

FIG. 2 is a front elevational view of the protective cover device;

FIG. 3 is an elevational view, in partial cross-section, showing the protective cover device of the present invention fitted over the hand and forearm of a user to cover a cast or bandage worn on the user's wrist and lower forearm;

FIG. 4 is a perspective view showing the protective cover device of the present invention worn over a user's foot and lower leg to cover a cast worn on the foot and lower leg;

FIG. 5 is an elevational view of the protective cover device showing another embodiment of the strap securing mechanism on the upper end of the cover device; and

FIG. 6 is an elevational view, in partial cross-section, showing the protective cover device of FIG. 5 and illustrating movement of the cinch strap relative to a securing buckle in order to adjustably close the open top end of the cover device.

Like reference numerals refer to like parts throughout the several views of the drawings.

### DETAILED DESCRIPTION OF THE PREFERRED EMBODIMENTS

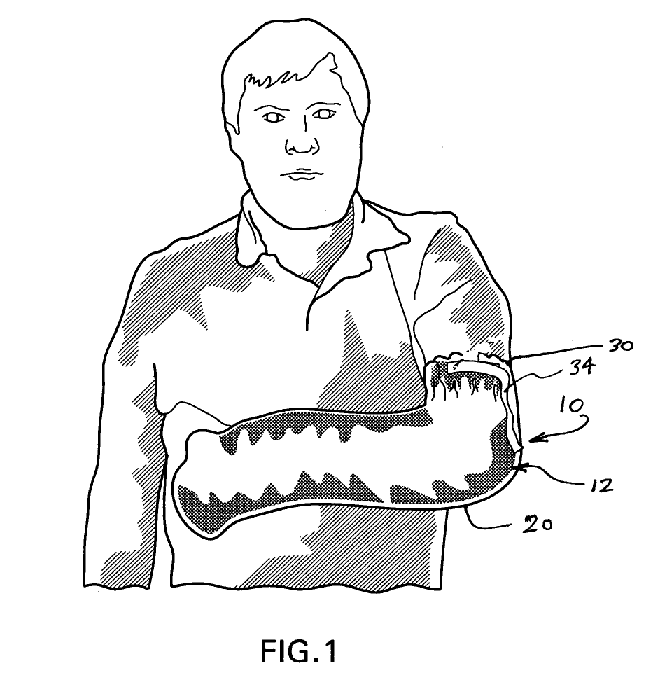

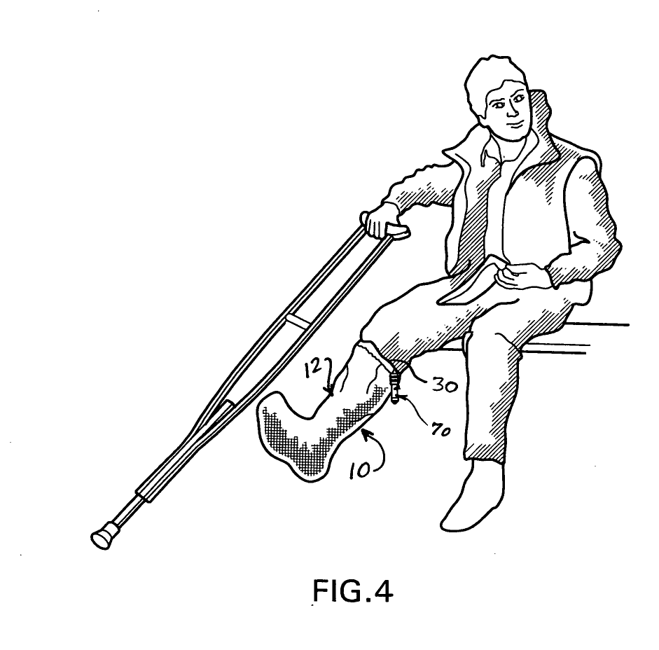

Referring to the several views of the drawings, the protective cover of the present invention is shown and is generally indicated as 10. The protective cover 10 is specifically adapted to be worn over a person's limb to shield a bandage, wound dressing or cast from exposure to water and moisture. The cast cover 10 includes a main body 12 formed and configured to cover a specific area of the body, such as a hand and at least a portion of an arm, as shown in FIGS. 1 and 3, or a foot and at least a portion of a leg, as shown in FIG. 4.

The main body of the cover device has a shell 20 that defines the overall size and shape of the cover device. The shell 20, forming the main body 12, has an open top end 30 communicating with an interior 40 that is surrounded by the shell. The shell 20 is formed of a flexible, water resistant and air breathable material. The interior 40 of the cover device includes an upper interior chamber 42 and a lower interior chamber 44. The open top end 30 communicates with the upper interior chamber 42 and is sized and configured to permit passage of a person's limb and a cast, bandage or dressing (all indicated by 50 in FIG. 3) therethrough and into the upper 42 and lower 44 interior chambers. The lower interior chamber 44 may be formed to generally conform with the shape of a hand, foot or other body part and is sufficiently sized to accommodate a cast, bandage or dressing 50 that is worn on the person's hand and arm or foot and leg.

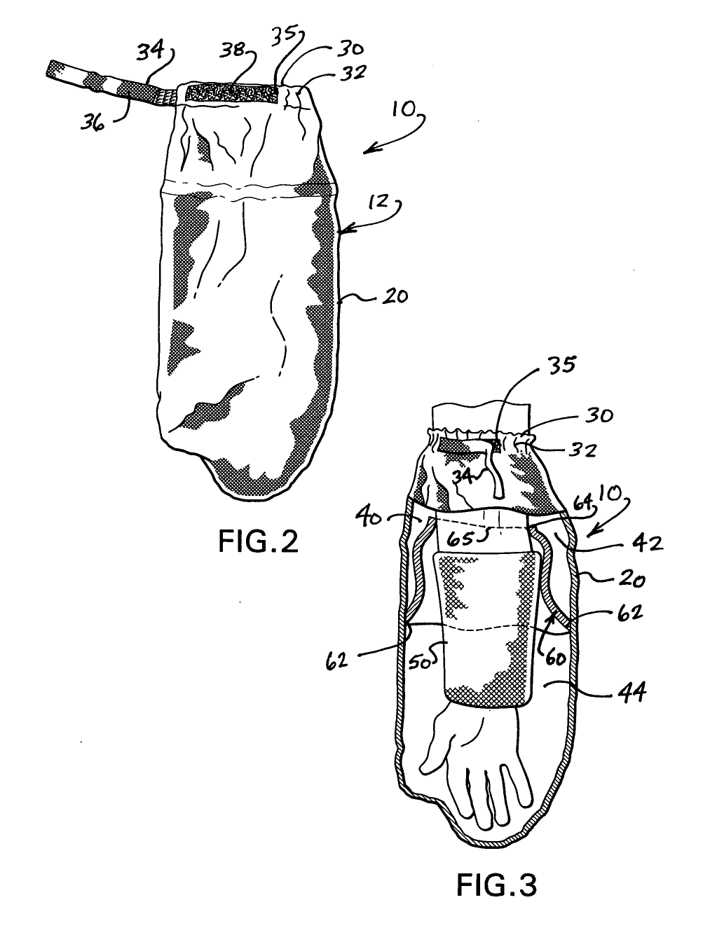

Once the cover device 10 is pulled over the person's limb, to cover the cast, bandage or dressing 50, as seen in FIGS. 1, 3 and 4, the open top end 30 is closed against the person's limb to discourage entry of water into the interior 40 of the cover device. In a first embodiment, as shown in FIGS. 1-3, the open top end 30 is fitted with an elastic band 32 within the shell material to partially close the open top end when in a relaxed state. The elastic band 32 can be stretched to enlarge the open top end to allow insertion and removal of the person's hand, or foot and limb therethrough. The embodiment shown in FIGS. 1-3 further includes a cinch strap 34 having a first end 35 permanently attached to the shell 20 of the cover device 10. An inner face of the cinch strap 34 is fitted with one component 36 of a hook and loop fastener on a free end segment and is adapted for releasable engagement with a corresponding component 38 of a hook and loop fastener on an opposite end segment of the strap, adjacent the open top end 30. When the cover device 10 is worn on the user, as seen in FIG. 3, the cinch strap 34 is pulled to close the top end 30 snug against the person's limb, above the cast, bandage or dressing 50 and is held secured by the mating attachment of the hook and loop fasteners 36, 38. This provides a first barrier against entry of water into the interior 40 of the cover device 10.

The cover device further includes an interior annular cuff 60 that separates the upper 42 and lower 44 interior chambers. The cuff 60 is secured about its outer periphery 62 to an inner side of the shell. The cuff 60 includes a central opening 64, surrounded by an elastic band 65, that allows passage of the person's hand or foot, and a portion of the arm or leg, therethrough and into the lower interior chamber 44, as seen in FIG. 3. When properly worn, the elastic band 65 surrounding the central opening 64 of the cuff snugly engages the user's limb above the cast, bandage or dressing, as shown in FIG. 3. This provides a second barrier against water entry into the lower interior chamber 44.

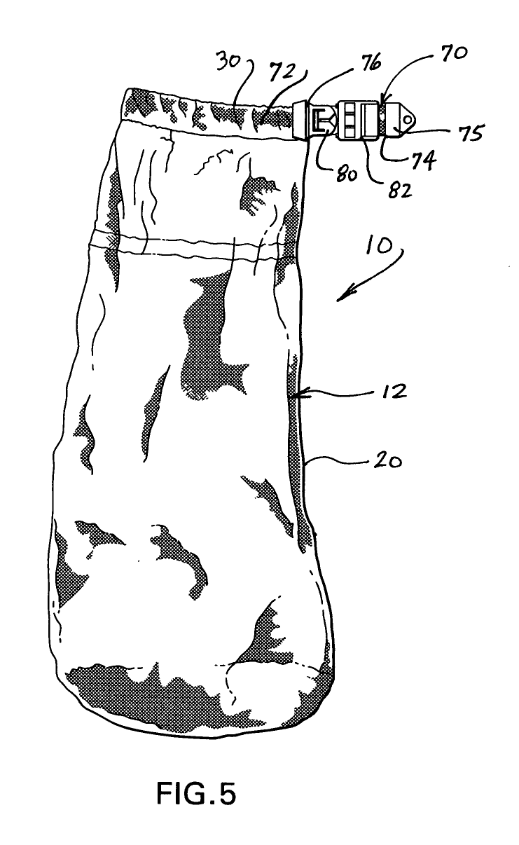

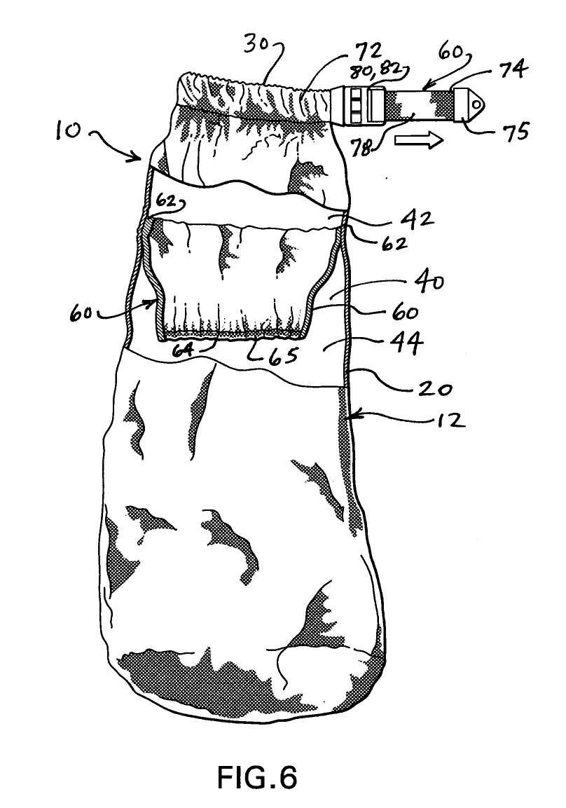

FIGS. 4-6 illustrate a second embodiment of the cinch strap 70. In this particular embodiment, the cinch strap 70 extends through a channel 72 surrounding the open top end 30. Opposite ends of the strap 70 remain exterior of the channel including a first end 74 fitted with a stop member 75 and an opposite end 76 fitted with a male component 80 of a buckle. A female component 82 of the buckle is slideably captivated on a first end zone 78 of the strap 70 between the stop member 75 and the channel 72 surrounding the open top end. To close the top end 30 snug about the limb of the user, the female component 82 of the buckle is secured to the male component 80 and, next, the first end 74 of the strap 70 is pulled, as indicated by the directional arrow in FIG. 6, to partially close the open top end 30 until it is snug about the user's limb.

In a preferred embodiment, both the shell 20 and the interior annular cuff 60 are formed of a water resistant, air breathable material. An example of a water resistant and air breathable material that is suitable for manufacture of the cover device, in particularly, the interior and annular cuff, is TYVEK from the DuPont company.

While the present invention has been shown and described in accordance with several preferred and practical embodiments, it is recognized that departures from the instant disclosure and fully contemplated within the spirit and scope of the present invention which is not to be limited except as defined in the following claims as interpreted under the Doctrine of Equivalence.

What is claimed is:

1. A protective cover device for covering a cast on a limb of a user when bathing, said cover device comprising:
a shell formed in a generally elongate configuration and including an outer surface, an inner surface, a closed bottom, a closed side wall structure surrounding an interior of said shell, and an open top end communicating with said interior;
an interior annular cuff secured to the inner surface of said shell between said open top end and said closed bottom to define a lower interior chamber for receiving at least a portion of the user's limb and the cast, and an upper interior chamber between said interior annular cuff and said open top end, and said interior annular cuff structured and disposed for snuggly engaging the user's limb beyond the cast to close said lower interior chamber with the at least a portion of the user's limb and the cast protectively contained therein;
an elastic band surrounding an open top end of said shell, and said elastic band structured and disposed for partially closing the open top end when in a relaxed state, and further structured and disposed for allowing insertion and removal of the user's limb and the cast into the interior of said shell; and
a cinch strap with a releasable securing mechanism for closing the open top end of said shell against the user's limb to discourage entry of water and moisture into the interior of said shell.

2. The device as recited in claim 1 wherein said shell is formed of a flexible, water resistant, and air breathable material.

3. The device as recited in claim 2 wherein said interior annular cuff is formed of the flexible, water resistant, and air breathable material.

4. The device as recited in claim 3 wherein said releasable securing mechanism is a hook and loop fastener.

5. The device as recited in claim 4 wherein a first component of said hook and loop fastener is on a distal end segment of said cinch strap and a corresponding component of said hook and loop fastener is located on an opposite end segment of said cinch strap.

6. A protective cover device for covering a cast on a limb of a user when bathing, said cover device comprising:
a shell formed in a generally elongate configuration and including an outer surface, an inner surface, a closed bottom, a closed side wall structure surrounding an interior of said shell, and an open top end communicating with said interior;
an interior annular cuff secured to the inner surface of said shell between said open top end and said closed bottom to define a lower interior chamber for receiving at least a portion of the user's limb and the cast, and an upper interior chamber between said interior annular cuff and said open top end, and said interior annular cuff structured and disposed for snuggly engaging the user's limb beyond the cast to close said lower interior chamber with the at least a portion of the user's limb and the cast protectively contained therein; and
a cinch strap extending through a channel surrounding the open top end of said shell, and said cinch strap being structured and disposed for closing the open top end of said shell against the user's limb to discourage entry of water and moisture into the interior of said shell.

7. The device as recited in claim 6 wherein said shell is formed of a flexible, water resistant, and air breathable material.

8. The device as recited in claim 7 wherein said interior annular cuff is formed of the flexible, water resistant, and air breathable material.

9. The device as recited in claim 8 wherein said cinch strap includes opposite end segments that remain exterior of said channel including a first end segment, a second end segment, and a releasable securing mechanism on said opposite end segments for holding the open top closed against the user's limb.

10. The device as recited in claim 9 wherein said releasable securing mechanism comprises:
a female component of a releasable buckle on said first end segment of said cinch strap; and
a male component of the releasable buckle on said opposite second end segment of said cinch strap for releasable, interlocked attachment with said female component.

11. The device as recited in claim 10 wherein said female component is structured to slide along said first end segment of said cinch strap, and said device further comprising a stop member on a distal end segment of said cinch strap for limiting movement of said female component.
